(19) United States
(12) Patent Application Publication
(10) Pub. No.: US 2008/0242944 A1
(43) Pub. Date: Oct. 2, 2008

(54) METHOD AND SYSTEM FOR FACILITATING ERROR FREE SCAN ADMINISTRATION

(75) Inventor: Neelam Sharma, Bangalore Karnataka (IN)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/693,861

(22) Filed: Mar. 30, 2007

(51) Int. Cl. A61B 5/00

(52) U.S. Cl. 600/300

(57) ABSTRACT

A method and system for facilitating error free scan is disclosed herein. The system for preventing wrong scan administration to a patient comprises: an identifier associated with a patient for identifying at least one patient parameter. The patient parameter may be configured to indicate patient information such as pregnancy or the presence of metallic implants. An interrogator is provided for automatically interrogating the identifier to obtain the at least one patient parameter during one or more predefined stages when the patient is in proximity of a scanner. A controller is operatively coupled to the interrogator for generating a wrong scan signal based on the communicated information.

### FIELD OF THE INVENTION

This invention relates generally to automated workflows in hospitals or other healthcare environments, and more particularly relates to a method and system for facilitating an error free scan administration.

### BACKGROUND OF THE INVENTION

Medical errors cause huge money and time losses and may damage a patient's health. Some of the examples of medical errors are scanning a pregnant patient using an X-ray scanner or any other scanning device using X-ray radiations and/or injection of a radioactive substance into the patient before scanning (e.g., as in Positron Emission Tomography). X-ray radiations in case of pregnant women can cause a hazard to the unborn child. Similarly scanning of a patient having metallic implants using a Magnetic Resonance Imaging (MRI) scanner may be hazardous to the patients due to the influence of a strong magnetic field generated by the scanner on the metallic implants.

The cause of a large number of medical errors can generally be attributed to a manual or partially automated process employed in a medical facility or hospital for loading a medical information concerning a patient and checking the same before performing each medical procedure. Considering an existing workflow in a hospital, loading of the medical information is often a manual process. One of the methods for an operator who performs the scanning is to get medical information about the patient by asking the patient about his medical information or by fetching a medical record corresponding to the patient that may be entered manually by a person while the patient is admitted in the hospital, or may be stored as part of the patient's medical record.

The primary limitation in the conventional process involved in procuring and loading the medical information, is a possible occurrence of a medical error. For example, the medical error can involve a wrong entry of medical information such as a pregnant patient as “not pregnant”. It is also possible to have a medical error while manually interpreting the medical information. The operator may wrongly interpret the information provided by the medical information record or patient, for example, even though the medical information indicates the patient as “pregnant”, the operator may by mistake interpret the same as “not pregnant”.

Another limitation in using the conventional process is the selection of the patient or a mix up of the medical information of one patient with the medical information of another patient. The wrong selection of the patient can lead to a wrong scanning procedure being administered on the patient. For example if medical information about a patient having metallic implants is mixed with the medical information of a patient who does not have any metallic implants, wrong scanning procedure may be prescribed for the patient.

Thus there exists a need to provide a system and method for preventing a wrong scan administration to a pregnant women, a patient with metallic implants, or a patient with another type of medical condition that is incompatible with a scan being taken. A workflow should be provided to facilitate an error free scanning to patients.

### SUMMARY OF THE INVENTION

The above-mentioned shortcomings, disadvantages and problems are addressed herein which will be understood by reading and understanding the following specification.

The present invention provides a system for preventing wrong scan administration to a patient. The system comprises: (a) an identifier associated with a patient for identifying at least one patient parameter; (b) an interrogator for automatically interrogating the identifier to obtain the at least one patient parameter when the patient is in proximity of a scanner; and (c) a controller operatively coupled to the interrogator, wherein the controller is configured to generate a wrong scan signal based on the at least one patient parameter. In another embodiment, a method of facilitating error free scanning of a patient includes: (a) providing a patient with an identifier indicating a condition of the patient; (b) automatically reading the condition of the patient from the identifier when the patient moves into proximity with a scanner; and (c) generating an error signal if the condition of the patient is incompatible with a scan being taken using the scanner.

In another embodiment, a method of facilitating error free scanning to a patient is disclosed. The method comprises: (a) providing a patient with an identifier indicating at least one of patient information about pregnancy or metallic implants; (b) triggering a first alarm signal, the first alarm signal being generated by a controller associated with a scanning room; and (c) triggering a second alarm signal, the second alarm signal being generated by a controller associated with the scanner. The first and second alarm signals are generated upon detection of a pregnant patient or a patient with a metallic implant.

In yet another embodiment, a method of preventing wrong scan administration to a patient is described. The method comprising the steps of: (a) acquiring patient information about pregnancy and metallic implants; (b) assigning a patient parameter corresponding to the acquired patient information; the patient parameter being configured to indicate at least one of patient information about pregnancy or metallic implants; (c) providing an identifier to the patient, the identifier being associated with a patient parameter; (d) interrogating the identifier at a plurality of pre defined stages; and (e) controlling scan administration to the patient based on the interrogated information.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of a system for preventing wrong scan administration to a patient as described in an embodiment of the invention;

FIG. 2 is a flow chart illustrating a method of facilitating error free scanning as described in an embodiment of the invention;

FIG. 3 is a flow chart illustrating a method of facilitating error free scanning as described in an embodiment of the invention;

FIG. 4 is flow chart illustrating exemplary steps of a method of preventing wrong scan administration to the patient as described in an embodiment of the invention;

FIG. 5A is a workflow illustrating a patient entry to a hospital in implementing error free scanning as described in an embodiment of the invention;

FIG. 5B is a workflow illustrating patient entry to a scanning room in implementing error free scanning as described in an embodiment of the invention; and

FIG. 5C is a workflow illustrating a patient scanning in implementing error free scanning as described in an embodiment of the invention.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

In various embodiments, a method and system for facilitating error free scanning is provided. The concept is explained in reference to medical imaging field. However it shall be noted that, the application of the concept of the invention need not be limited to this. Rather it shall be extended to variety of application, where some checking measures need to be taken, before performing an operation. The applications may include mechanical, electrical applications as well, wherein some precautionary measures to be taken before proceeding with different steps.

In various embodiments, a method and system for preventing wrong scan administration to a patient is provided. The system includes an identifier associated with the patient for identifying at least one patient parameter or condition of the patient, such as whether the patient is pregnant, has a metallic implant that would be affected by the magnetic field produced by an MRI scanner, or has some other medical condition that would be incompatible with a particular type of scanning procedure. The system also includes an interrogator for automatically interrogating the identifier to obtain the patient parameter or condition when the patient is in proximity of the scanner, such as when the patient walks or is otherwise moved into a scanning room or when the patient walks or is otherwise moved into proximity with a scanner. The system further includes a controller coupled to the interrogator which is configured to generate a wrong scan signal based on the condition of the patient. In one embodiment, the wrong scan signal is used to activate an audible or visible alarm to the operator of the scanner to notify the operator that the patient has a condition that is incompatible with being scanned by the scanner, thereby giving the operator an opportunity to cancel the scan, or conduct the scan in such a way as to be compatible with the condition of the patient. In another embodiment, the wrong scan signal is provided to the scanner itself to inhibit or disable the scanner from conducting the scan. In certain embodiments, the method includes providing an identifier for identifying a pregnant patient and a patient with metallic implants and controlling the administration of scanning using the information conveyed by the identifier to prevent harm to the patient due to his or her condition. The application of the invention need not be limited to pregnant patients or patients with metallic implants, further it could be extended different situations such as a patient who undergoes a PET scan and is allergic to some kinds of dyes which need to be injected before performing the scanning operation.

In an embodiment, the invention uses a plurality of identifiers like RFID tag, barcodes, magnetic strips, wireless devices such as Bluetooth, Infrared, etc, but need not be limited to these and the patient is checked for their status corresponding to pregnancy, metallic implants or any other condition that could affect scanning at different stages such as before entering into a scanning room, before initializing the scanning operation.

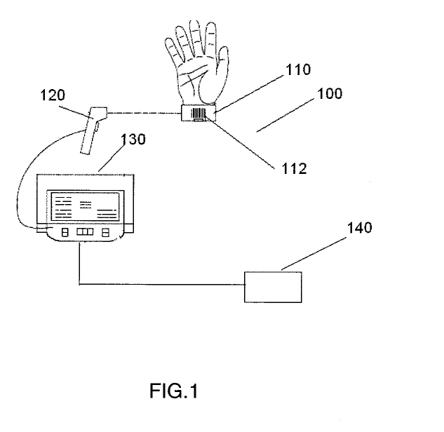

FIG. 1 is a schematic diagram of a system for facilitating error free scanning administration as described in an embodiment of the invention. The system 100 includes an identifier 110, an interrogator 120 and a controller 130. The identifier 110 is provided in association with a patient, such as by being attached to the patient by a wrist-strap as in FIG. 1, or by otherwise being coupled to the patient. In one embodiment particularly suitable to a patient having a metallic implant, the identifier 110 is embedded into the body of the patient, such as in a subcutaneous device inserted under the skin of the patient and capable of communicating through the skin. Thus, the identifier 110 may be associated with the patient by being coupled thereto either externally or internally. The identifier 110 identifies at least one condition or parameter of the patient, as shown by reference numeral 112. In one embodiment, the patient parameter 112 is configured to indicate at least one of patient information about pregnancy or the presence of a metallic implant. The patient parameter 112 could be determined based on the patient status that includes patient information about pregnancy or metallic implants and also based on the additional information such as nature of scanner or scanning room etc and the medical procedure that the patient is intending to undergo. The patient information could be obtained from the patient or could be achieved from prior records stored in any memory device or in the hospital's computer system. For example, a patient parameter 112 is assigned to a patient when the patent is detected as being pregnant or is detected as having a metallic implant. The patient parameter 112 is indicated through the identifier 110.

The identifier 110 is associated with the patient for indicating the patient parameter 112. The identifier could be a bar code strip, an RFID tag, a magnetic strip or any wireless transponder such an Infrared or Bluetooth device, but need not be limited to these examples. The identifier could be provided on the patient in the form of a label, tag or string that could be attached to or hung from the body. However the identifier could be located in proximity to the patient as well. For example the wireless device or transponder need not be provided on the patient, but could be provided in proximity to the patient such as being coupled to a patient table supporting the patient. The embodiment illustrated shows the identifier provided on the wrist of a patient, but it could be provided on any convenient part of the body or in proximity to the patient. The identifier 110 is provided to the patient upon entering the patient into the hospital or when the patient is scheduled for scanning. Before assigning the patient parameter 112 and the identifier 110 a plurality of patient information is acquired. In one embodiment, the patient information includes patient status information indicative of whether the patient is pregnant or the patient has metallic implants. This information may be recorded by an operator. If the patient is scheduled for scanning and is detected as pregnant or has metallic implants, he or she is provided with an identifier. The identifier 110 will carry information about the patient status indicating whether the patient is pregnant or has metallic implants. In an embodiment the identifier 110 may include additional information such as information about the scanning room, nature of scanner, etc. Also the identifier 110 may be configured to carry patient identification information that could be used for tracking the patient in a medical facility or may be used for scheduling different medical procedures. In an embodiment an RFID tag is used as an identifier as it will automatically emits signals so that the identifier will be read automatically without any human intervention.

The interrogator 120 is provided for interrogating the identifier 110 to obtain the patient parameter 112 or patient condition information. The interrogator 120 is automatically configured to interrogate the identifier 110 to obtain the patient parameter 112 when the patient is in proximity of the scanner. The proximity of the scanner may include patient entering a scanning room or positioning patient for a scan in the scanner. The interrogator 120 may be selected from an RFID reader, bar code reader, a magnetic strip reader or a wireless receiver. The interrogator 120 may be based on the corresponding identifier associated with the patient. The interrogator 120 is configured to obtain at least one patient parameter upon entering the patient to a scanning room. The interrogator 120 is further configured to obtain at least one patient parameter patient being positioned in a scanner. The interrogator 120 can be directly or indirectly associated with the scanner or scanning room. But it should be not be limited so and as per the requirement the interrogator may be located wherever there is a need to check the patient status.

In an embodiment at least two interrogators are provided. One interrogator is provided at the scanning room. As soon as the patient enters the scanning room the interrogator reads the information and generates control signals or alarms based on the read information. The first interrogator may be provided at the entrance of the scanning room so that the entry of the patient to the scanning room is restricted based on the information conveyed by the identifier. A second interrogator may be provided at the scanner. The second interrogator may be configured to control the operation of the scanner directly, or to generate an alarm or message to the operator using the scanner.

The interrogated or read information may be communicated to a corresponding controller 130. Generally a controller 130 is provided corresponding to each interrogator, but there could be an integrated single controller to generate control signals based on the information obtained from the interrogators. In an example two controllers are provided, the first controller is associated with the first interrogator and the second controller is associated with the second interrogator. The controller is configured to generate a wrong scan signal when the patient parameter indicates that the patient is pregnant and/or when the patient parameter indicates that the patient has metallic implants. The wrong scan signal could act as an inhibit signal for inhibiting the scan of patient when applied to the scanner. The controller 130 can be any processor that will be capable of generating a control signal based on the information conveyed to the controller. The controller 130 may be provided with a memory (not shown). The memory can have the details of the scanning room and details of the scanner. For example the controller 130 may be fed with an information that the scanner is an MR scanner, when a patient is positioned for scanning, the identifier 110 is read and based on the read information and the information saved in the controller 130, the controller 130 will be able to generates more accurate result. Also this will give the flexibility of using the same or similar identifiers for identifying the pregnant patients, as well patients with metallic implants or another condition that effects the suitability of performing different types of scanning. For example, the same RFID frequency may be used to identify the pregnant patients and patients with metallic implants. In this event the identifier is further provided with information about the medical object and that can be compared with the corresponding information available with the controller 130. Once the RFID tag is read, the controller 130 will get the information that the patient is pregnant, or the patient has metallic implants or another condition that effect the suitability of performing scans. The controller 130 is further provided with the information about the nature of scanning room or scanner. Based on the nature of scanner and the read information, which includes patient information as well as the additional information such as information about the nature of scanning room or scanner, the controller 130 generates controlling signals. In some embodiment the interrogator 120 may be provided with an integrated controller or the controller may be provided with an integrated interrogator so that detecting the identifier and controlling the system may be performed by a single unit.

As mentioned earlier, the second interrogator is provided with a scanner. Once the patient is positioned on the scanner or positioned in front of a detector, the second interrogator reads the identifier. A second controller is associated with a scanner for receiving the identified information and controlling the operation of the scanner. The controller may generate a signal to control the initialization of the scanning operation. This could be achieved by providing a checking before initializing the scanning operation by the scanner to determine whether any control signal is received by the scanner. Alternately the control signal may be used to control the X-ray source or detector or MR field generator so that the scanning operation is not performed. In an embodiment the system 100 may be configured to control or restrict the operation of the scanner. In some embodiments, this restriction automatically inhibits the scanner from performing the scan. In other embodiments, this restriction alerts the operator to the potential problem and thereafter relies on further commands from the operator to determine its operation.

In an embodiment the system is further provided with an alarm device 140. The alarm device 140 is operably coupled to the controller and is configured to generate an alarm signal based on the wrong scan signal generated by the controller 130. There could be different alarm devices that could be associated with each controller. For example, the alarm-could be generated at the scanning room level or at the scanner level. The alarm can be in any operator-perceptible form such as an audio or visual signal.

In an embodiment the system 100 is configured to control the entry of a patient to a scanning room. Each scanning room is provided with an interrogator. The nature of the interrogator may depend on the nature of the identifier provided to the patient. Before the patient enters into the scanning room, the interrogator reads the identifier and if it does not identify any hazardous signal then the patient is allowed to enter into the scanning room. The hazardous signal indicates whether the scanning procedure is harmful to the patient. The patient may be prevented from entering into the scanning room upon identification of a hazardous signal either automatically by, for example, controlling an electro-magnetic lock, or manually by an operator.

For convenience, the following paragraphs describe an exemplary system that is designed to prevent an X-ray examination of a pregnant woman to prevent harmful X-ray exposure to the fetus, or to prevent an MRI examination of a patient having a metallic implant. However, it should be understand that pregnancy or the presence of metallic implants are just two potential patient parameters or conditions that could cause a wrong scan to be administered to the patient, and the invention is not limited to either or both of these conditions. Rather, the present invention extends to preventing the wrong scan administration due to other patient parameters, such as, for example, the prevention of PET scans to patients who are allergic to particular types of contrast agents, or to the prevention of X-ray scans to patients who have already received prior X-ray exams and should not undergo further X-ray scans unless necessary, in order to limit their exposure.

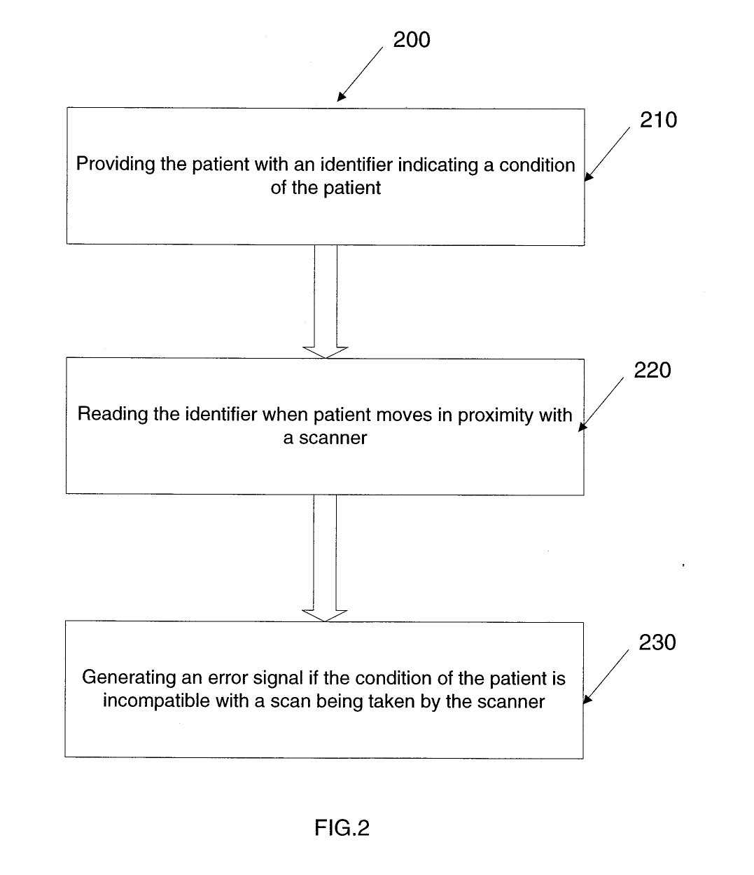

FIG. 2 is flow chart illustrating a method 200 of facilitating error free scanning as described in an embodiment of the invention. At step 210, the patient is provided with an identifier indicating a condition of the patient. The various conditions may include the patient status about pregnancy or information about metallic implants provided on the patient or any other relevant information. In an example if the patient is pregnant she may be provided with an RFID tag and if the patient has a metallic implant he or she may be provided with an identifier configured to indicate the same. At step 220, the identifier is read when the patient moves into proximity with a scanner. The identifier is read to identify the condition of the patient. The proximity of the scanner may include events such as patient entering into the scanning room or positing the patient for scanning or any other similar instances wherein scanning of a patient could be hazards to the patients. At step 230, an error signal is generated if the condition of the patient is incompatible with a scan being taken using the scanner. Error signal may be generated in different situations such as the patient is pregnant and if the scanner produces X-rays. Similarly an error signal could be generated if the patient has a metallic implant and the scanner is a magnetic resonance scanner. The error signal may be further used to generate an alarm signal to an operator of the scanner. Also the error signal may be applied to the scanner for inhibiting the scanner from scanning the patient.

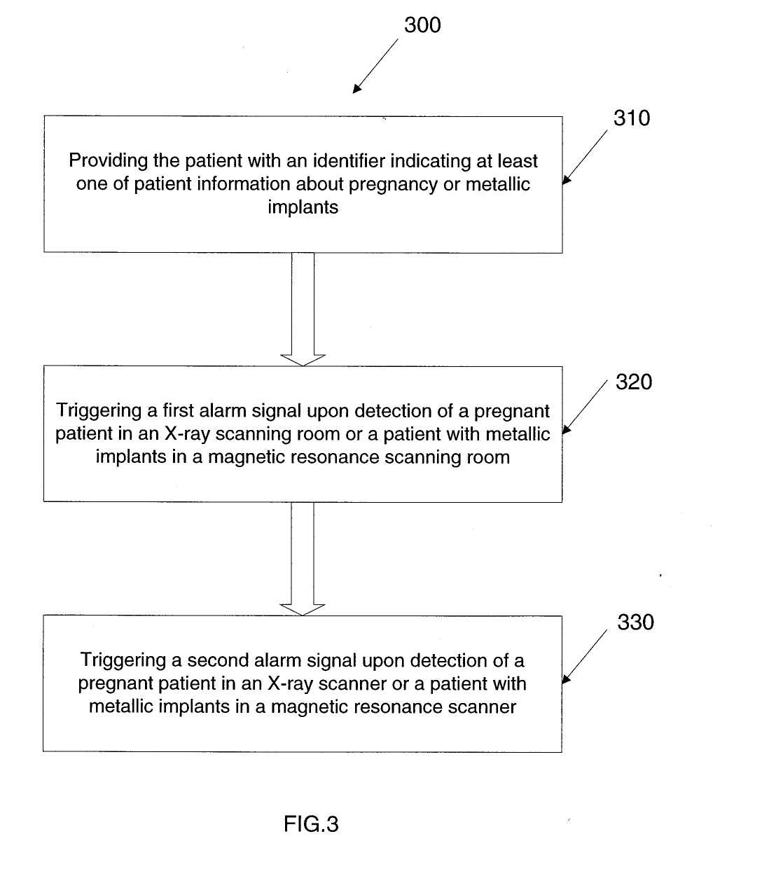

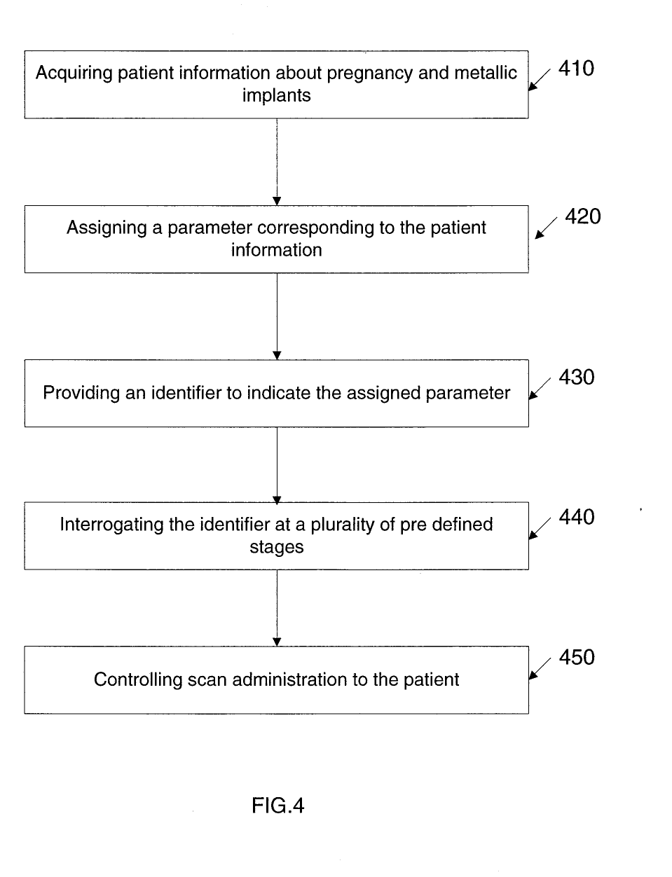

FIG. 3 is flow chart illustrating a method 300 of facilitating error free scanning as described in an embodiment of the invention. At step 310, the patient is provided with an identifier. The identifier may be associated with the patient by being coupled thereto either externally or internally. The identifier identifies at least one condition or parameter of the patient such as whether the patient is pregnant or the patient has metallic implants. The identifier may be assigned to the patient while entering into the hospital or when the patient is scheduled for scanning. In another embodiment, as in the case of a patient having metallic implants, the patient is provided with the identifier at an earlier time, such as when the implants were made. At step 320, a first alarm signal is generated when a pregnant patient enters into an X-ray scanning room or a patient with metallic implants enters into an MR scanning room. The alarm signal may be generated based on the information read from the identifier and/or based on additional information such as nature of the scanning room. The first alarm signal is generated at the scanning room level. This could be used to control the admission of a patient in different scanning rooms based on the patient status or patient condition. At step 330, a second alarm signal is generated at the scanner level if a pregnant patient is detected on or placed for an X-ray scanning and a patient with metallic implants is positioned for an MR scanning. The alarm signal may be generated based on the information read from the identifier and/or based on additional information such as nature of the scanner. This may be used to control the operation of the scanner and/or to alert the operator to a potential problem. The various steps involved in the method are explained in reference to FIG. 4

FIG. 4 is flow chart illustrating exemplary steps of a method of preventing wrong scan administration to the patient as described in an embodiment of the invention. At step 410, a plurality of patient information is acquired. The patient information includes patient status regarding pregnancy and metallic implants. The patient information may also include the patient identification information or additional information such a nature of scanner or scanning room or any other relevant information. The patient information may be acquired from patient, server or from any memory device. At step 420, a patient parameter is assigned to patients based on the patient information. The patient parameter is configured to indicate at least one of patient information about pregnancy or metallic implants. At step 430, an identifier is provided with the patient. The identifier is configured to indicate the patient parameter. At step 440, the identifier is interrogated at a plurality of pre-defined stages. Some examples include patient entering into the scanning room or positioning the patient for scanning. The interrogator reads the identifier and communicates the same to a controller. The interrogator may also interpret the information and generate a control signal. At step 450, the scan administration is controlled by the control signal. The control signal may control the initialization of the scanner. The controlling operation is performed such that no pregnant patient is allowed in an X-ray scanning room/positioned for an X-ray scanning and no patient with metallic implants is allowed inside an MR scanning room/positioned for an MR scanning.

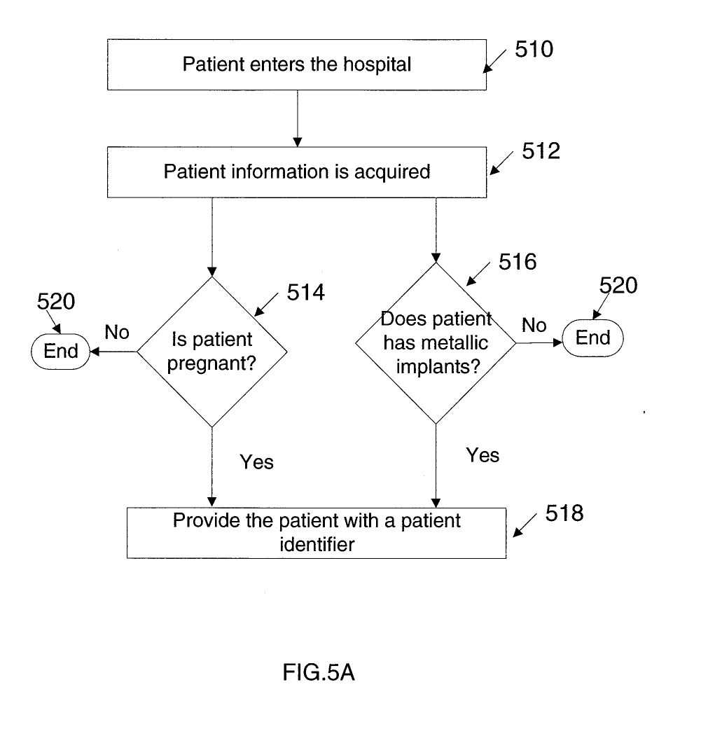

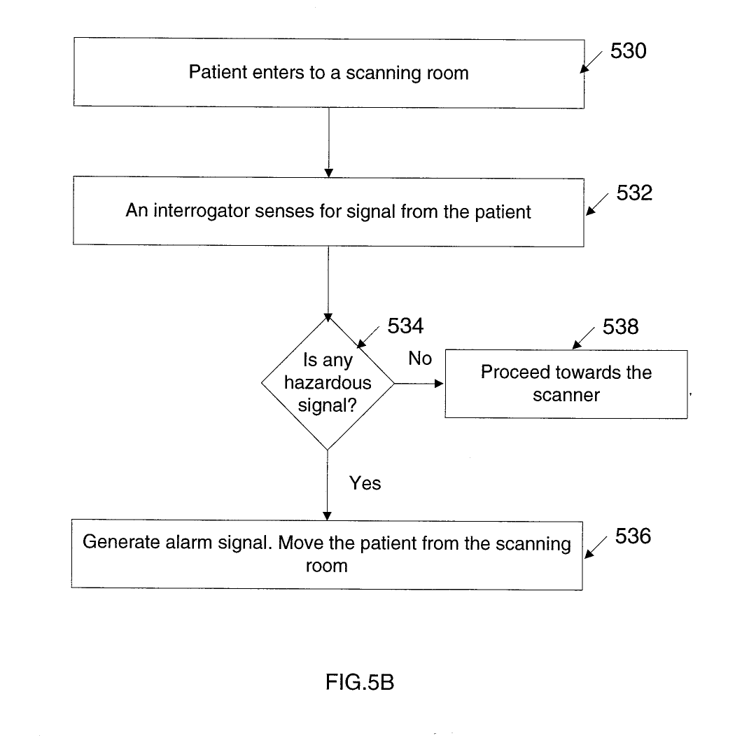

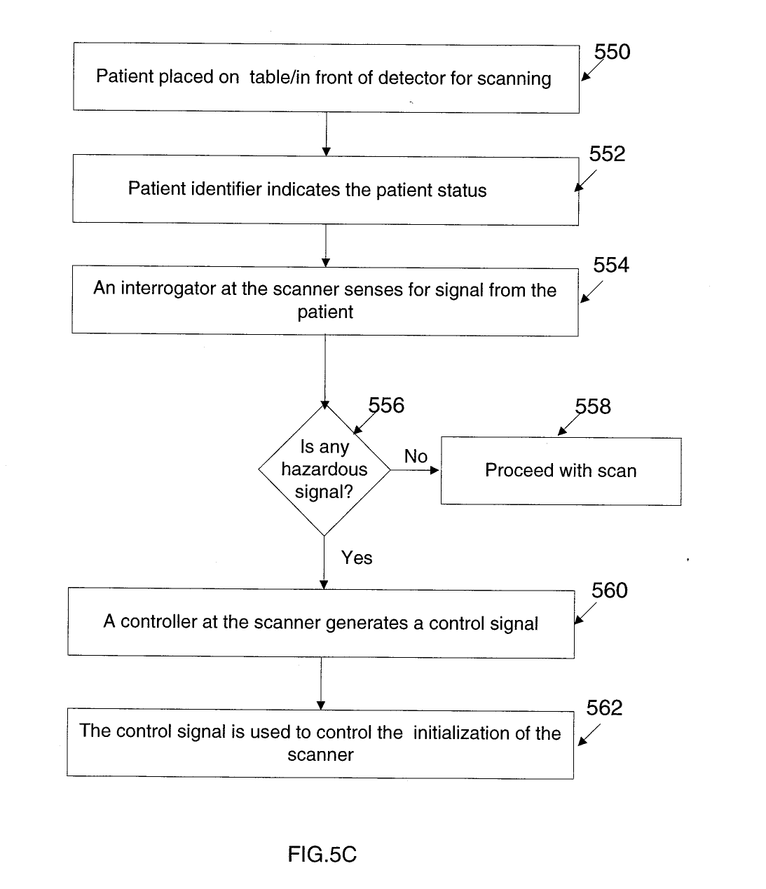

FIG. 5A is a workflow illustrating a patient entry to a hospital in implementing the error free scanning as described in an embodiment of the invention. At step 510 the patient enters into a hospital. The process of facilitating error free scanning is initiated as and when the patient enters into the hospital. Alternately the workflow may start from a medical procedure wherein the patient is scheduled for scanning. At step 512 a plurality of patient information is acquired. The patient information will include patient status about pregnancy and metallic implants. The operator may ask the patient and record this information or the system or operator may fetch this information from a server or a memory device. Other than the information about pregnancy and metallic implants, several other information may also be acquired and stored. For example patient identification information, medical procedures the patient is planning to undergo etc may be of relevance to the context. At step 514 the system checks whether the patient is pregnant and at step 516 the system checks whether the patient has metallic implants. The steps 514 and 516 can be performed in any order or, in other embodiments, only one of steps 514 and 516 may be performed. Once the patient is detected as pregnant, she may be checked for metallic implants, so that she should not be allowed to both X-ray as well as MR scanning room. If the patient is identified with metallic implants and she needs to be checked whether she is pregnant. At step 518, the patient is detected with metallic implants, or detected as pregnant or both are assigned with an identifier. The patient detected as pregnant or detected with metallic implants may be provided with same kind of identifiers. For example, both may be provided with RFID tags of same frequency. In that event, the identifier should be stored with additional information about the nature of scanner and a scanning room etc. The identifier may also be stored with patient identification information which could be used in tracking the patient in the hospital and for scheduling the patient for various medical procedures. If the patient is detected as not pregnant and does not have any metallic implants the patient is not assigned with any identifier and the workflow is ended as shown in step 520.

FIG. 5B is a workflow illustrating patient entry to a scanning room in implementing the error free scanning as described in an embodiment of the invention. At step 530, the patient enters into a scanning room. The patient is provided with an identifier indicating the status of the patient. For example whether the patient is pregnant or the patient has metallic implants. At step 532, an interrogator provided at the scanning room senses or reads the identifier. For example if the identifier is an RFID tag, the tag will be emitting signals and the corresponding interrogator, an RFID reader, will automatically sense the emitted signal. At step 534 the system checks whether any hazardous signal being generated. Any signal generated by the interrogator based on the identifier is referred as hazardous signal as they could be hazardous to the pregnant patients or patients with metallic implants. At step 536, if any hazardous signal is sensed, the system will generate an alarm signal to alert the operator and the patient is moved out of the scanning room quickly. If no hazardous signals are sensed the patient is allowed to proceed for the scanning as shown in step 538.

FIG. 5C is a workflow illustrating patient scanning in implementing the error free scanning as described in an embodiment of the invention. At step 550, the patient is placed on a patient table or in front of the detector for scanning. At step 552, the identifier provided with the patient indicates the patient status. For example, in case of a patient wearing an RFID tag emits a signal continuously. At step 554, an interrogator provided in association with the scanner reads the identifier. For example, the interrogator senses the signal emitted by the RFID tag. At step 556, the scanner checks whether any hazardous signal is being detected. The hazardous signal may correspond to an indication that the patient who is ready to undergo scanning is pregnant or has metallic implants. If no hazardous signal is detected, the patient is allowed to proceed with the scanning as shown at step 558. If the system detects any hazardous signal, then at step 560, a control signal being generated by a controller associated with the scanner. At step 562, the control signal is configured to control the initialization of the scanner. For example in case of an X-ray scanner the control signal may disable the X-ray source or detector. In case of MR scanner, the control signal may be used to control the magnetic field generator. Alternatively, the control signal may be used to activate an alarm to notify the operator of a potential hazard, and the operator then decides how or whether to proceed.

Some of the advantages of the invention include providing an improved patient safety mechanism while the patient undergoes scanning. The invention automatically facilities error free scan administration by using an RFID associated with the patient. However other identifiers could be used to identify the patients with minimal manual intervention. This will enable the hospital authorities to delegate certain works to non-trained staff. This will avoid hazard to a fetus while scanning. The invention will reduce or avoid the manual errors that could occur while administering a scanning.

The above-description of the embodiments of the system 100 and the method 200 and 300 has the technical effect of facilitating an error free scan administration. This will allow the scanning workflow in a medical environment to be automatically controlled to minimize human interventions and the medical errors caused by operators.

Thus various embodiments of the invention describe a system and method for facilitating an error free scan administration. Also in an embodiment a method of performing a scanning using the concept of error free handling is disclosed.

In another embodiment, the invention is used to prevent wrong scan administration for people passing through a security checkpoint. For example, the identifier may include a tag or label coupled to a person passing through a security checkpoint at an airport, and the identifier indicates the person has a pacemaker. The checkpoint includes a metal, explosive, radioactive or other detector that emits a signal or field that may be hazardous to the passenger due to his pacemaker. The detector includes an interrogator that automatically interrogates the identifier to obtain information about the passenger's condition (i.e., having a pacemaker). A controller uses the information about the pacemaker to activate an alarm to the security personnel indicating that the passenger should be processed without going through the detector, or to activate a gate to prevent the passenger from walking into the detector, or to inhibit the operation of the detector. The passenger is thereby protected from the potential hazard to his health.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

I claim:

1. A system for preventing wrong scan administration to a patient, comprising:
(a) an identifier associated with a patient for identifying at least one patient parameter;
(b) an interrogator for automatically interrogating the identifier to obtain the at least one patient parameter when the patient is in proximity of a scanner; and
(c) a controller operatively coupled to the interrogator, wherein the controller is configured to generate a wrong scan signal based on the at least one patient parameter.

2. A system as in claim 1, wherein the identifier is configured to identify a patient parameter indicative of whether the patient is pregnant.

3. A system as in claim 1, wherein the identifier is configured to identify a patient parameter indicative of whether the patient has a metallic implant.

4. A system as in claim 1, wherein the identifier is an RFID tag.

5. A system as in claim 1, wherein the identifier is selected from a group consisting of a barcode, a magnetic strip, and a wireless transponder.

6. A system as in claim 1, wherein the interrogator is associated with a scanner.

7. A system as in claim 1, wherein the interrogator is associated with a scanning room.

8. A system as in claim 4, wherein the interrogator is an RFID reader.

9. A system as in claim 5, wherein the interrogator is selected from a group consisting of a barcode reader, a magnetic strip reader and a wireless receiver.

10. A system as in claim 1, wherein the interrogator is configured to obtain the at least one patient parameter upon the patient entering into a scanning room.

11. A system as in claim 1, wherein the interrogator is configured to obtain the at least one patient parameter upon the patient being positioned in a scanner.

12. A system as in claim 1, wherein the interrogator includes a first interrogator and a second interrogator, the first interrogator being associated with a scanning room and the second interrogator being associated with a scanner.

13. A system as in claim 12, wherein the controller includes a first controller and a second controller, the first controller being associated with the first interrogator and the second controller being associated with the second interrogator.

14. A system as in claim 13, wherein the controller is configured to generate the wrong scan signal when the patient parameter indicates the patient is pregnant.

15. A system as in claim 13, wherein the controller is configured to generate the wrong scan signal when the patient parameter indicates the patient has a metallic implant.

16. A system as in claim 1, wherein the wrong scan signal is a scan inhibit signal for inhibiting a scan of the patient when applied to a scanner.

17. A system as in claim 1, wherein the system further comprises an alarm device, wherein the alarm device is configured to generate an operator-perceptible alarm in response to the wrong scan signal.

18. A method of facilitating error free scanning of a patient, comprising:
(a) providing a patient with an identifier indicating a condition of the patient;
(b) automatically reading the condition of the patient from the identifier when the patient moves into proximity with a scanner; and
(c) generating an error signal if the condition of the patient is incompatible with a scan being taken using the scanner.

19. The method of claim 18, wherein the providing step comprises providing a patient with an RFID tag and the automatically reading step comprises automatically reading the condition of the patient from the RFID tag.

20. The method of claim 18, wherein the condition indicates the patient is pregnant, and the error signal is generated if the scanner produces X-rays.

21. The method of claim 18, wherein the condition indicates the patient has a metallic implant, and the error signal is generated if the scanner is a magnetic resonance imaging scanner.

22. The method of claim 18, further comprising the step of providing an alarm to an operator of the scanner in response to the error signal.

23. The method of claim 18, further comprising the step of applying the error signal to the scanner to inhibit the scanner from scanning the patient.

24. A method of facilitating error free scanning to a patient, comprising:
(a) providing a patient with an identifier indicating at least one of patient information about pregnancy or metallic implants;
(b) triggering a first alarm signal, the first alarm signal being generated by a controller associated with a scanning room; and
(c) triggering a second alarm signal, the second alarm signal being generated by a controller associated with the scanner;
wherein the first and second alarm signals are generated upon detection of a pregnant patient or a patient with a metallic implant.

25. A method as in claim 24, wherein the step of triggering a first alarm signal comprises: reading the identifier by an interrogator associated with the scanning room and generating the first alarm signal based on the read information.

26. A method as in claim 24, wherein the step of triggering a first alarm signal comprises: reading the identifier by an interrogator associated with the scanning room and generating the first alarm signal based on the read information and the nature of the scanning room.

27. A method as in claim 24, wherein the step of triggering a second alarm signal comprises: reading the identifier by an interrogator associated with the scanner and generating the alarm signal based on the read information.

28. A method as in claim 24, wherein the step of triggering a second alarm signal comprises: reading the identifier by an interrogator associated with the scanner and generating the alarm signal based on the read information and the nature of the scanner.

29. A method as in claim 24, wherein the step of triggering a first alarm signal further comprises: controlling admission of a pregnant patient to an X-ray scanning room and a patient with metallic implants to an MR scanning room.

30. A method as in claim 24, wherein the step of triggering a second alarm signal further includes controlling the operation of a scanner upon positioning a pregnant patient in proximity to an X-ray scanner and positioning a patient with metallic implants in proximity to an MR scanner.

31. A method of preventing wrong scan administration to a patient, comprising the steps of:
(a) acquiring patient information about pregnancy and metallic implants;
(b) assigning a patient parameter corresponding to the acquired patient information; the patient parameter being configured to indicate at least one of patient information about pregnancy or metallic implants;
(c) providing an identifier to the patient, the identifier being associated with a patient parameter;
(d) interrogating the identifier at a plurality of pre defined stages; and
(e) controlling scan administration to the patient based on the interrogated information.

32. A method as in claim 31, wherein the step of acquiring patient information comprises: obtaining information from the patient, fetching the information a server, or obtaining the information from prior records or a memory device.

33. A method as in claim 31, wherein the step of controlling further comprises: controlling admission of a patient with a metallic implant in an MR scanning room and admission of a pregnant patient in an X-ray, CT scanning room.

34. A method as in claim 31, wherein the step of controlling further comprises: controlling operation of an MR scanner when the patient is detected with a metallic implant and an X-ray scanner when the patient is pregnant.
